(19) United States
(12) Patent Application Publication
(10) Pub. No.: US 2014/0316263 A1
(43) Pub. Date: Oct. 23, 2014

(54) TEMPORARY DIALYSIS VASCULAR ACCESS CONDUIT OCCLUSION DEVICE AND METHOD OF DEMONSTRATING ARTERIAL COMMUNICATION DURING DIAGNOSTIC DIALYSIS FISTULOGRAPHY

(71) Applicant: Timothy P. Murphy, Providence, RI (US)

(72) Inventor: Timothy P. Murphy, Providence, RI (US)

(21) Appl. No.: 13/868,354

(22) Filed: Apr. 23, 2013

(51) Int. Cl. A61B 6/00; A61M 29/02; A61M 1/36

(52) U.S. Cl. CPC A61B 6/481; A61M 1/3653; A61M 29/02
USPC 600/431; 606/191; 606/198; 606/194

(57) ABSTRACT

The current invention describes a device and method for occlusion of a blood vessel to enable contrast-enhanced radiographic images to be obtained proximal to the blood vessel to be occluded, into the direction of flow and upstream from the entry site of the device into the blood vessel.

### FIELD

This patent application references and claims the priority date of provisional patent application No. 61/640031, EFS ID No. 12657296, “Temporary Dialysis Vascular Access Occluder Device and Method of Demonstrating Arterial End of Dialysis Vascular Access During Diagnostic Dialysis Fistulography”, (Timothy Patrick Murphy, inventor), filed and received Apr. 30, 2012. The current patent application incorporates the invention of the provisional patent application.

The present invention relates to methods and devices for performing surgical and radiological procedures, and in particular to methods and devices for temporarily blocking outflow from a dialysis access fistula and thereby permitting contrast opacification and radiographic imaging of the dialysis access fistula communication with the proximal artery.

### BACKGROUND

There are over 200,000 people in the U.S. with end-stage renal disease (ESRD) undergoing dialysis. For most of these, dialysis is accomplished by filtering the blood (hemodialysis). Hemodialysis requires high-volume blood flow (3-5 liters/minute) which usually is achieved by using a vascular graft or fistula (“vascular access”), which is usually created in the forearm or upper arm.

Dialysis grafts or fistulas are not permanently effective and many demonstrate problems with dialysis after they have been in use for some time. Such problems often result in inadequate or inefficient hemodialysis, and this will usually prompt a diagnostic evaluation to determine if the graft or fistula can be fixed, or whether a new access for dialysis will need to be created.

The standard method for evaluation of failing dialysis vascular access is fistulography, which is done by inserting a needle or catheter into the fistula, injecting radiopaque contrast material, and obtaining radiographic images. Usually, the needle or catheter is introduced into the fistula in the direction of the presumed venous communication or outflow. Most stenoses are toward that end of the vascular access and if such stenoses are discovered, then they can often be treated with balloon angioplasty (PTA) or stent placement using the same access.

However, the arterial inflow should always be evaluated as part of a radiographic examination of a dialysis vascular access conduit. This poses problems since the flow through the fistula can be rapid enough that refluxing contrast into the direction of the blood flow can be difficult. Often, physicians will manual compress the outflow from the vascular access temporarily while injecting contrast by squeezing or pressing on the palpable segment of the fistula downstream from the access. This maneuver is technically difficult, and often requires more than one attempt to adequately visualize the arterial anastomosis. It is also physically awkward, required the physician to extend their arm under the image intensifier, and often exposes their fingers to direct radiation during the acquisition of images, and their arm to near-proximity scatter radiation.

### SUMMARY

The devices and methods disclosed herein generally involve using of the invention to block outflow of a dialysis vascular access conduit to permit, thereby permitting radiographic contrast injected to flow backward, against the normal direction of blood flow. If radiographic contrast is injected at sufficient volume and rate, it will flow into the communication with the feeding artery, thereby permitting radiographic imaging of said arterial communication to be obtained.

In one aspect, the device has an initial conformation suitable for percutaneous insertion into a dialysis vascular access fistula, and a second conformation with a distal end that can be expanded within the dialysis access fistula to block blood flow through the fistula while still maintaining control of the distal end using an extension that maintains communication with the external world.

In another example, the dialysis vascular access occluder has a side port suitable for contrast injection.

In another example, the dialysis vascular access occluder can be recaptured and removed from the body.

A component of the present invention is the method of using a temporary intraluminal device that can be expanded to block blood flow through the dialysis vascular access fistula, so that contrast injected proximal to the distal end of the device flows backwards against the normal direction of blood flow within the dialysis vascular access fistula, and across the arterial communication, thereby allowing radiographic images to be done of the arterial communication.

### BRIEF DESCRIPTION OF THE DRAWINGS

FIG. 1 depicts one embodiment of the invention, in this case comprising two components.

FIG. 2 depicts an embodiment of the invention as in FIG. 1, mated together and prepared for use. In this illustration, the device is in its initial conformation.

FIG. 3 is a depiction of an embodiment of the invention deployed to its second conformation.

FIG. 4 is a first serial exemplary depiction of an embodiment of the invention in use.

FIG. 5 is a second serial exemplary depiction of an embodiment of the invention in use

FIG. 6 is a third serial exemplary depiction of an embodiment of the invention in use

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Devices and related methods are disclosed that generally involve the use of the invention during radiographic examination of dialysis vascular access conduits, whereby outflow from the dialysis vascular access conduit is blocked, so that contrast injected using the same access to deliver the device would flow backwards against the normal direction of blood flow through the dialysis vascular access conduit, and if injected in sufficient volume and rate, across the arterial communication with the dialysis vascular access conduit, permitting radiographic images of the arterial communication with the dialysis vascular access conduit to be obtained.

In one embodiment, a temporary dialysis vascular access conduit occlusion device is provided that can be introduced through standard percutaneous access in its initial conformation, then a distal component can be expanded within the dialysis vascular access conduit using mechanical means controlled by the operator, and then when contrast is injected contrast flows backward or retrograde to the normal direction of flow through the dialysis vascular access conduit, such that if contrast is injected at sufficient rate and volume it will eventually pass into the feeding artery, allowing radiographic images to be obtained of the communication with the feeding artery.

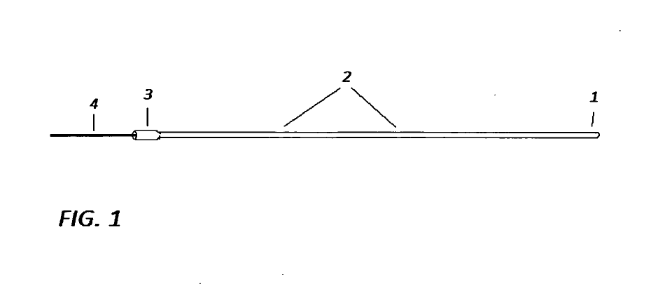

FIG. 1 depicts one embodiment of the invention. In this example, the device consists of two components, the occlusion device 1 and its delivery sheath 2, which are depicted here separated for illustration purposes. The occlusion device 1 has a distal end 3 that has a variable diameter, being in its initial collapsed conformation when introduced in the body, but then expanded as illustrated here when positioned appropriately within the vascular access conduit to cause blockage of blood flow through the conduit. In this embodiment, the distal end 3 of the occlusion device 1 is comprised of an expandable network of wires, covered in part or in whole by a membrane that is essentially impermeable to blood flow, so that when expanded in its second conformation essentially all blood flow through the dialysis vascular access conduit ceases. The occlusion device 1 also has a proximal tether or wire 4 that maintains communication between the distal end 3 and the outside world, thereby enabling control of the distal end 3 by the operator. The delivery sheath 2 has an expanded proximal end 5, in this example depicted as a hub, to facilitate manipulation, and a distal end 6 that in this example envelopes the occlusion device permitting entry into the body through standard vascular sheaths.

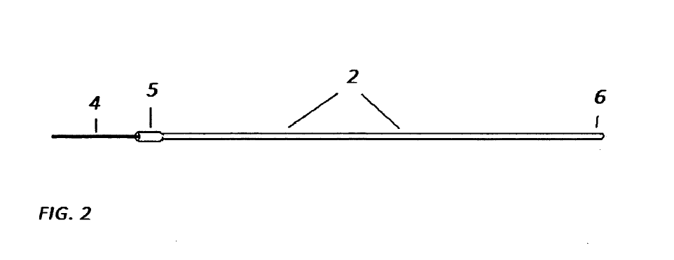

FIG. 2 illustrates the two components mated together by insertion of the occlusion device 1 into the delivery sheath 2, as an example of how this embodiment of the invention is prepared for use. The distal end 3 of the device is concealed within its covering sheath 2, and is maintained in its initial conformation in this example by being constrained within an outer catheter or sheath 2. The outer catheter or sheath is of suitable caliber and length to be introduced through a second standard vascular access sheath 7, and in this embodiment has an expanded proximal end such as a hub 5 or other component to assist manipulation of the device when loaded in this fashion for entry into the body. A tether or wire 4 extends proximally and maintains communication with the outside world so that the distal end of the device can be controlled by the operator. In this embodiment, the distal end of the device is made of material with elasticity or shape-memory, such that when the constraining apparatus is released it will immediately assume its second conformation. Those skilled in the art will readily appreciate other methods of achieving expansion of the distal end 3.

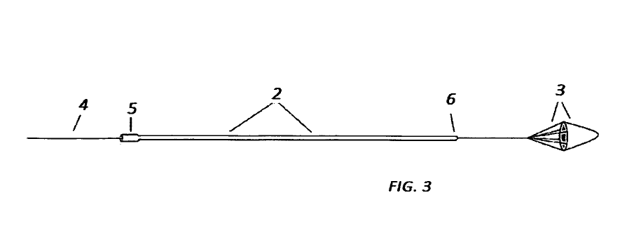

FIG. 3 is an illustration of one embodiment of the invention in its second conformation. This embodiment of the device is transformed when positioned appropriately in the body from its initial to its second conformation by manipulation or release of the constraining apparatus, in this example done by retraction of the covering sheath 2 proximally relative to the distal end 3 of the occlusion device.

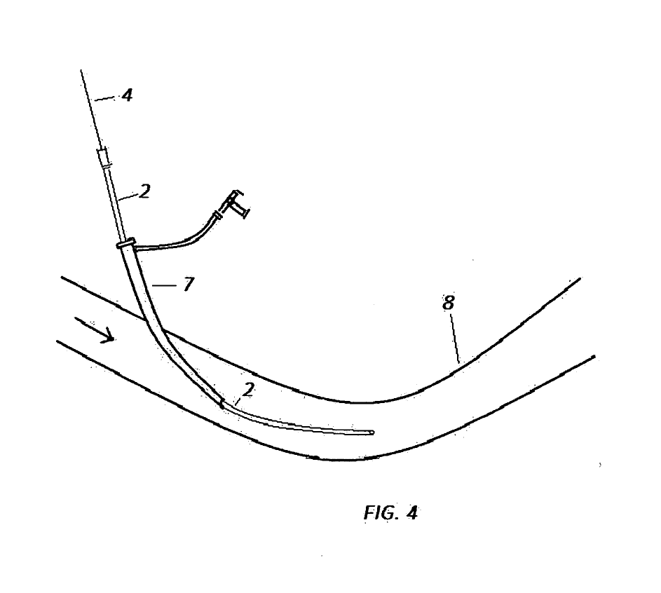

FIG. 4 is a first serial depiction of one embodiment of the invention in use, in this case introduced through an existing vascular sheath 7, positioned so that its distal end of the delivery sheath 2 exits the vascular sheath and resides in the lumen of dialysis vascular access conduit 8, in this representation shown in longitudinal section.

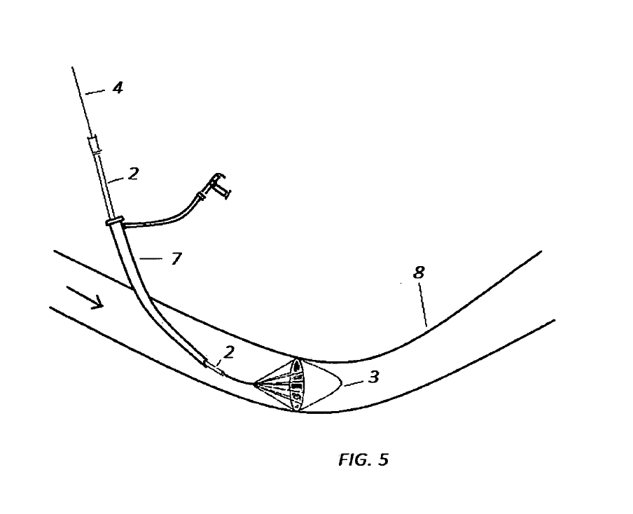

FIG. 5 is a further serial depiction of one embodiment of the invention in use. The distal end 3 of the occlusion device has in this example been unsheathed by retraction of the delivery sheath 2 relative to the distal end 3 of the occlusion device, which is maintained stationary relative to the delivery sheath by maintaining control of the tether or wire 4. Once unsheathed in this example, the distal end 3 of the occlusion device component expands and occupies essentially all of the cross-sectional area of the lumen of the dialysis vascular access conduit 8. The arrow indicates the direction of blood flow through the dialysis vascular access conduit as depicted in this example of use.

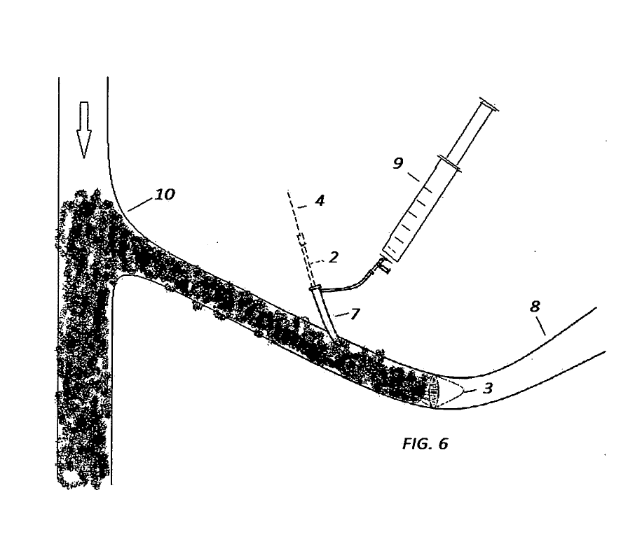

FIG. 6 is a third serial depiction of one embodiment of the device in use, where radio-opaque contrast material, depicted here in a syringe 9, is injected either through a more proximal component of the device, such as for example its constraining sheath 2 or vascular sheath 7, while the device is in its second conformation, resulting in filling of the blood vessel proximal to the distal end of the device, and even proximal to the location of entry of the vascular sheath in the blood vessel, to allow radiographic images obtained essentially simultaneously to demonstrate the communication with the artery 10 that supplies blood flow to the dialysis vascular access conduit 8. The arrow in this example depicts the direction of blood flow in supplying artery. In this depiction, gray shading illustrates radiographic contrast material in the lumen of the dialysis vascular access conduit 8, flowing against the usual direction of blood flow and across the communication with the supplying artery 10. After use in this manner, the distal end is re-constrained to its initial conformation, in this example by advancing the delivery sheath, and removed from the body.

What is claimed is:

1. A device used for temporary occlusion of blood flow in a dialysis vascular access conduit comprising a first conformation adaptable to pass through an opening in a vessel and a second conformation adaptable to conform to an inner wall of a vessel.

2. A device used for temporary occlusion of blood flow in a dialysis access graft of claim 1 comprising a distal end and a proximal end; and when positioned appropriately in the body, has a distal component that can be transformed into a second configuration by mechanical means.

3. A device used for temporary occlusion of blood flow in a dialysis access graft of claim 2 that in its initial conformation, the distal section introduced into the body has an essentially uniform outer diameter.

4. A device used for temporary occlusion of blood flow in a dialysis access graft of claim 1 that in its second conformation has a distal component that occupies essentially all of the cross-sectional area of the dialysis access conduit.

5. A device used for temporary occlusion of blood flow in a dialysis access graft of claim 1 that comprises a distal component that when expanded to its second conformation causes essentially cessation of blood flow in the dialysis vascular access conduit.

6. A device used for temporary occlusion of blood flow in a dialysis access graft of claim 1 that has a distal component that is transformed from its initial configuration to its second configuration by self-expansion using shape-memory materials.

7. A device used for temporary occlusion of blood flow in a dialysis access graft of claim 1, that has a distal component that is transformed from its initial configuration to a second configuration by release or activation of a constraining mechanism.

8. A device used for temporary occlusion of blood flow in a dialysis access graft of claim 1, that has a distal component that is transformed from its initial configuration to a second configuration by exposure to body temperature.

9. A device used for temporary occlusion of blood flow in a dialysis access graft of claim 1 that has a distal component that is transformed from its initial configuration to its second configuration by foreshortening of a component of its distal end relative to a component of its proximal end.

10. A device used for temporary occlusion of blood flow in a dialysis access graft of claim 1 that has a distal component is transformed from its initial configuration to its second configuration by expansion of an angioplasty balloon.

11. A device used for temporary occlusion of blood flow in a dialysis access graft of claim 1 that comprises a distal component affixed to a tether apparatus at its proximal end that maintains contact with the outside world during its use.

12. A device used for temporary occlusion of blood flow in a dialysis access graft of claim 1 that can be converted from its second conformation to its first conformation by mechanical means.

13. A method performed during radiographic examination of dialysis access conduits that comprises enhancing radiographic contrast opacification of communications between arteries and veins by using a temporary device introduced into the dialysis access conduit to restrict the flow of blood through it.

14. A method of claim 13 that comprises introducing a temporary device into the dialysis access conduit in an initial conformation.

15. A method of claim 13 that comprises introducing a temporary device into the dialysis access conduit while maintaining control of the device by a wire, catheter, tether, or other direct means.

16. A method of claim 13 that comprises expanding a distal component once positioned properly in the dialysis conduit.

17. A method of claim 13 that comprises injection of contrast and obtaining of radiographic images of the arterial communication with a dialysis access conduit while the temporary vascular occlusion device is in its second conformation in the dialysis vascular access conduit.

18. A method of claim 13 that comprises transformation of the temporary dialysis vascular access occlusion device from its second conformation to its initial conformation after radiographic images are completed.

19. A method of claim 13 that comprises removal of the temporary dialysis occlusion device from the dialysis access conduit and from the body after obtaining of radiographic images of the arterial communication with the dialysis vascular access conduit after transformation of the distal end from its second to its first conformation.

20. A method of claim 13 that comprises temporarily blocking nearly all blood flow through the dialysis vascular access conduit with an temporary occlusion device.

21. A method of claim 23 that comprises injection of contrast proximal to the entry site of the vascular access used for contrast injection, against the direction of flow, aided by occlusion of the distal outflow with a temporary device.

22. A method of claim 21 that comprises injection of contrast proximal to the entry site of vascular access used for contrast injection, against the direction of flow, by occlusion of the distal outflow, in order to visualize the connection between the artery and the dialysis vascular access conduit or vein in a dialysis access conduit.
